(19) United States
(12) Patent Application Publication
(10) Pub. No.: US 2023/0201093 A1
(43) Pub. Date: Jun. 29, 2023

(54) A PERSONAL CARE COMPOSITION BASED ON TITANIUM OXIDE AND A CROSSPOLYMER OF ADIPIC ACID AND NEOPENTYL GLYCOL

(71) Applicant: Conopco, Inc., d/b/a UNILEVER, Englewood Cliffs, NJ (US)

(72) Inventors: Lin WANG, Shanghai (CN); Bo XU, Shanghai (CN); Shangchun YI, Shanghai (CN)

(73) Assignee: Conopco, Inc., d/b/a UNILEVER, Englewood Cliffs, NJ (US)

(21) Appl. No.: 17/928,327

(22) PCT Filed: May 21, 2021
(86) PCT No.: PCT/EP2021/063567

§ 371 Date: Nov. 29, 2022

(30) Foreign Application Priority Data
Jun. 19, 2020 (WO) PCT/CN2020/097189
Aug. 6, 2020 (EP) 20189734.5

(51) Int. Cl. A61K 8/29; A61K 8/85; A61Q 1/02

(52) U.S. Cl. CPC A61K 8/29; A61K 8/85; A61Q 1/02; A61K 2800/621; A61K 2800/43; A61K 2800/412

(57) ABSTRACT

The present invention relates to a personal care composition. In particular, the present invention relates to a personal care composition for blurring benefits. Accordingly, the present invention discloses a personal care composition comprising: a) titanium di-oxide; and b) 8 to 60% by weight of a cross-polymer comprising adipic acid and neopentyl glycol.

### FIELD OF THE INVENTION

The present invention relates to a personal care composition. In particular, the present invention relates to a personal care composition for blurring benefits.

### BACKGROUND AND PRIOR ART

Appearance of an individual changes with his/her age. These changes become visible in various skin attributes such as, wrinkles formation, pores, age spots etc. These attributes cause imperfection in the appearance of skin. To counter this and in order to look younger, people use various skin care products.

Skin care products containing particles and pigments are particularly helpful in this regard. Scientists have found out that, particles and pigments when applied in a layer onto the skin, interact with incoming light and modify the distribution of reflected light. The reflected light changes the appearance of the skin. Therefore, skin care products containing right selection of particles and pigments which can provide betterment in the appearance of the skin is a subject matter of interests. This motivates personal care industries to develop new products comprising particles and pigments for enhancing appearance of skin.

One of the benefits provided by aforesaid products, is improving the appearance by providing blurring benefit. It is achieved by reducing the contrast between adjacent areas of skin by a topical layer of particles. Particles scatter incoming light in different directions uniformly. It reduces contrast between adjacent points on the skin, which causes the imperfections to be less prominent over the skin background. Some of the prior art in same technical field are cited below.

US 2007/0243220 (Revlon Consumer Products Corp, 2007) describes a cosmetic composition comprising a crosslinked a polyester polyol and pigments in a cosmetically acceptable carrier for improving aesthetics of the composition It further describes aesthetic of the composition comprising crosslinked polyester polyol is comparable to composition containing silicon elastomer.

WO 2015/178007 (Shiseido Company Ltd, 2015) describes a sunscreen product in which excessive whiteness due to titanium dioxide and zinc oxide are visually masked upon skin application. To mask the excessive whiteness that would otherwise result from the oxides, the sunscreen products contain multilayer-type encapsulations containing pigments. When the sunscreen product is applied to the skin, the capsules break, and the pigments are released to counter the excessive whiteness due to titanium dioxide and zinc oxide.

Although, the prior art documents describe cosmetic compositions comprising particles and pigments, however, they are not efficacious in providing significant blurring benefit. Moreover, compositions with relatively high particle loading lead to a patchy appearance on skin, which is not preferred by the individuals.

Therefore, a personal care composition with improved with relatively low particle loading, which provides improved blurring benefit to skin and improve the appearance, remains to be desired.

### OBJECTS OF THE INVENTION

In view of the foregoing, it is an object of the present invention to provide a skin care composition.

It is another object of the present invention to provide a skin care composition for improving skin appearance.

It is yet another object of the present invention to provide a skin care composition that renders blurring effect on skin and thereby improves the skin appearance of individuals.

The present inventors have surprisingly found that a personal care composition comprising titanium dioxide having a particular particle size and a cross-polymer comprising adipic acid and neopentyl glycol in a particular amount provides enhanced blurring benefit and thereby improves skin appearance.

Thus, satisfying one or more of the above mentioned objects.

### SUMMARY OF THE INVENTION

In a first aspect, the present invention provides a personal care composition comprising;
(a) titanium di-oxide having a particle size in the range of 50 to 600 nm; and
(b) 8 to 60% by weight of a cross-polymer comprising adipic acid and neopentyl glycol.

In a second aspect, the present invention provides a method for improving the appearance of skin by blurring the imperfections on the skin comprising the steps of applying a composition according to the first aspect of the invention on the desired skin surface.

In a third aspect, the present invention provides the use of a composition according to the first aspect of the invention for blurring benefits on skin.

In a fourth aspect, the present invention provides the use of a composition comprising titanium di-oxide having a particle size in the range of 50 to 600 nm and 8 to 60% by weight of cross-polymer comprising adipic acid and neopentyl glycol for blurring benefits.

These and other aspects, features and advantages will become apparent to those of ordinary skill in the art from a reading of the following detailed description. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

### DETAILED DESCRIPTION OF THE INVENTION

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

By “A Personal Care Composition” as used herein, is meant to include a composition for topical application to the skin of mammals, especially humans. Such a composition may be generally classified as leave-on or rinse off but is preferably of the leave on type. The composition is formulated into a product which is applied to a human body specifically for improving appearance but may also be capable of providing cleansing, odor control or general aesthetics. The composition of the present invention can be in the form of a liquid, lotion, cream, foam, scrub, gel, or toner, or applied with an implement or via a face mask or a pad. Non-limiting examples of such compositions include leave-on skin lotions, creams, antiperspirants, deodorants, lipsticks, foundations, mascara, sunless tanners and sunscreen lotions. The composition of the present invention is preferably a leave-on composition. “Skin” as used herein is meant to include skin on the face and body (e.g., neck, chest, back, arms, underarms, hands, legs, buttocks and scalp) and especially to the sun exposed parts thereof.

The term “blurring” herein preferably refers to an optical effect induced by light interaction of inorganic particles on skin. The particles redistribute the light on skin surface to reduce contrast between adjacent areas thereby masking skin imperfections like wrinkles, age spots etc.

The term “particle size” herein typically means the apparent volume median diameter (D50, also known as x50 or sometimes d(0.5)) in non-aggregated state unless otherwise stated. For polydisperse samples having particulate with particle size less than 1 μm, particle size may be measured, for example, using dynamic light scattering (see international standard ISO 13321) with an instrument such as a Zetasizer Nano™ (Malvern Instruments Ltd, UK). For polydisperse samples having particulate with diameter at least 1 μm, particle size is measurable for example, by laser diffraction using a system (such as a Mastersizer™ 2000 available from Malvern Instruments Ltd) meeting the requirements set out in ISO 13320.

According to present invention there is provided a personal care composition comprising titanium di-oxide and 8 to 60% by weight of a cross-polymer comprising adipic acid and neopentyl glycol.

The titanium dioxide is preferably used as a scattering source of light in the composition. Without wishing to be limited by theory it is believed that titanium dioxide particles are distributed in a topical layer on skin after application of the composition and scatter the incoming lights in such a way that it induces optical blurring effect.

Preferably the amount of titanium di-oxide is in the range of 0.01 to 5% by weight, more preferably 0.01 to 2% by weight, even more preferably 0.05 to 2% by weight and most preferably 0.1 to 1% by weight of the composition.

Preferably the particle size of titanium di-oxide is in the range of 50 to 600 nm, more preferably in the range of 50 to 500 nm, further more preferably in the range of 1000 to 400 nm, even more preferably in the range of 200 to 400 nm and most preferably in the range of 250 to 400 nm.

Preferably the titanium di-oxide used in the present invention is coated with a hydrophobic coating. Preferably the hydrophobic coating may be selected from a fatty acid, a silicone and a metal oxide. Most preferred coating is selected from aluminium hydroxide and/or dimethicone.

The composition of the present invention also comprises 8 to 60% by weight of a crosspolymer comprising adipic acid and neopentyl glycol.

“Crosspolymer” as used herein refers to co-polymer which is made up of repeating units of one monomer or several monomers crosslinked with another monomer containing preferably at least three functional groups which is known as crosslinker. Different from non-crosslinked polymer, crosspolymer forms three-dimensional structure and hence exhibits good resistance to heat, solvent, wear, etc. Crosspolymers in general has been widely used in cosmetic products, as functional particles, film formers, thickeners, due to its unique chemical structure and excellent performance.

The cross-polymer used in the invention comprises adipic acid and neopentyl glycol. The crosspolymer used in the present invention preferably crosslinked with isopropyltriethylsilane. A particular example of a suitable and most preferred crosspolymer is adipic acid and neopentyl glycol crosslinked with isopropyltriethylsilane blended with a copolymer of vinyl pyrrolidone and vinyl acetate (INCI name adipic acid/neopentyl glycol crosspolymer).

Preferably the amount of cross-polymer is in the range of 10 to 60% by weight, more preferably 12 to 60% by weight and further more preferably 20 to 60% by weight of the composition.

In another preferred embodiments, the amount of cross-polymer comprises adipic acid and neopentyl glycol is present in the range of 15 to 50% by weight of the composition, more preferably 16 to 40% by weight and even more preferably 18 to 30% by weight of the composition.

Preferably, the weight ratio of the cross-polymer of cross-polymer comprises adipic acid and neopentyl glycol to the titanium di-oxide is in the range of 3000:1 to 1:1, more preferably 600:1 to 5:1, even more preferably 200:1 to 20:1 and most preferably 120:1 to 30:1.

The composition preferably additionally comprises one or more organic sunscreens. A wide variety of organic sunscreen is suitable for use in combination with the essential ingredients of this invention. Suitable UV-A/UV-B sunscreen include, 2-hydroxy-4-methoxybenzophenone, octyldimethyl p-aminobenzoic acid, digalloyltrioleate, 2,2-dihydroxy-4-methoxybenzophenone, ethyl-4-(bis(hydroxypropyl)) aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, 2-ethylhexyl-p-methoxycinnamate, butylmethoxydibenzoylmethane, 2-hydroxy-4-methoxybenzophenone, octyldimethyl-p-aminobenzoic acid and mixtures thereof. The most suitable organic sunscreens are 2-ethylhexyl-p-methoxycinnamate, butylmethoxydibenzoylmethane or a mixture thereof.

A safe and effective amount of organic sunscreen may be used in the compositions useful in the subject invention. The composition preferably comprises from 0.1% to 10%, more preferably from 0.1% to 5%, of organic sunscreen.

The composition of the invention preferably comprises a skin lightening agent. Vitamin B3 compounds (including derivatives of vitamin B3) e.g. niacin, nicotinic acid or niacinamide are the preferred skin lightening agent as per the invention, most preferred being niacinamide. Vitamin B3 compounds, when used, are preferably present in an amount in the range of 0.1 to 10%, more preferably 0.2 to 5% by weight of the composition.

The composition may comprise other beneficial skin care actives like retinol, retinyl esters, resorcinol, allantoin, ubiquinone, conjugated linoleic acid, 12-hydroxystearic acid or derivatives thereof. Of these the most preferred ones for inclusion in the composition of the invention are anti-aging actives like retinol or retinyl esters.

Compositions of the present invention will also include a cosmetically acceptable carrier which is a water and oil emulsion, which in certain embodiments may be water-in-oil emulsion. Preferred emulsions, however, are the oil-in-water variety.

Preferred hydrophobic material for use in the oil phase of such emulsions includes emollients such as fats, oils, fatty alcohols, fatty acids, soaps, silicone oils, synthetic esters and/or hydrocarbons.

Silicones may be divided into the volatile and non-volatile variety. Volatile silicone oils (if used) are preferably chosen from cyclic (cyclomethicone) or linear polydimethylsiloxanes containing from 3 to 9, preferably from 4 to 5, silicon atoms.

Non-volatile silicones useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5×10⁻⁶ to 0.1 m²/s at 25° C. Among the preferred non-volatile emollients useful in the present compositions are the polydimethyl siloxanes having viscosities from about 1×10⁻⁵ to about 4×10⁻⁴ m²/s at 25° C.

Specific examples of non-silicone emollients include stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate, and mixtures thereof.

Among the ester emollients are:
a) Alkenyl or alkyl esters of fatty acids having 10 to 20 carbon atoms. Examples thereof include isoarachidyl neopentanoate, isodecyl neopentanoate, isononyl isonanoate, cetyl ricinoleate, oleyl myristate, oleyl stearate, and oleyl oleate;
b) Ether-esters such as fatty acid esters of ethoxylated fatty alcohols;
c) Polyhydric alcohol esters. Butylene glycol, ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C₁-C₃₀ alcohols. An Example is pentaerythrityl tetraethylhexanoate;
d) Wax esters such as beeswax, spermaceti wax and tribehenin wax;
e) Sterols esters, of which cholesterol fatty acid esters are examples thereof;
f) Sugar ester of fatty acids such as sucrose polybehenate and sucrose polycottonseedate; or
g) mixtures of two or more of the foregoing (a) to (f).

Of particular use also are the C₁₂₋₁₅ alkyl benzoate esters sold under the Finsolv® brand.

Hydrocarbons which are suitable emollients include petrolatum, mineral oil, C₁₁-C₁₃ isoparaffins, polyalphaolefins, isohexadecane or a mixture thereof.

Amounts of water in the carrier may, for example, range from 1 to 99%, more preferably from 5 to 90%, even more preferably from 35 to 80%, optimally between 40 and 70% by weight of the personal care composition.

Other materials which can be included in the cosmetically acceptable carrier include solvents, humectants, thickeners and powders. Examples of each of these types of material, which can be used singly or as mixtures, are as follows: Solvents include ethyl alcohol, isopropanol, acetone, ethylene glycol monoethyl ether, diethylene glycol monobutyl ether, diethylene glycol monoethyl ether and mixtures thereof.

Humectants include those of the polyhydric alcohol-type. Typical polyhydric alcohols include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, glycerol, ethoxylated glycerol, propoxylated glycerol and mixtures thereof. The amount of humectant may range, for example, anywhere from 0.5 to 50%, more preferably between 1 and 15% by weight of the composition. Most preferred is glycerol (also known as glycerin). Amounts of glycerin may range, for example, from 0.5% to 50%, more preferably from 1 to 35%, optimally from 2 to 15% by weight of the composition.

A variety of thickening agents may be included in the compositions. Illustrative but not limiting are stearic acid, Acrylamide/Sodium Acryloyldimethyltaurate Copolymer (Aristoflex® AVC), Hydroxyethyl Acrylate/Sodium Acryloyldimethyltaurate Copolymer, Aluminum Starch Octenyl Succinate, Polyacrylates (such as Carbomers including Carbopol® 980, Carbopol® 1342, Pemulen TR-2° and the Ultrez® thickeners), Polysaccharides (including xanthan gum, guar gum, pectin, carageenan and sclerotium gums), celluloses (including carboxymethyl cellulose, ethyl cellulose, hydroxyethyl cellulose and methyl hydroxymethyl cellulose), minerals (including talc, silica, alumina, mica and clays, the latter being represented by bentonites, hectorites and attapulgites), magnesium aluminum silicate and mixtures thereof. Amounts of the thickeners may range, for example, from 0.05 to 10%, more preferably from 0.3 to 2% by weight of the composition.

Powders include chalk, talc, Fullers earth, kaolin, starch, gums, colloidal silica sodium polyacrylate, tetraalkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose and ethylene glycol monostearate.

The personal care composition of this invention is preferably a skin care composition. More preferably, the composition is preferably an antiperspirant composition or a face (except eye lids and lips) care composition. The skin care composition refers to a composition suitable for topical application to human skin, including leave-on and wash-off products. Preferably the term encompasses a fluid or liquid, and particularly a moisturizer rather than a make-up product. Most preferred are leave-on compositions. The term “leave-on” as used with reference to compositions herein means a composition that is applied to or rubbed on the skin and left thereon. The term “wash-off” as used with reference to compositions herein means a skin cleanser that is applied to or rubbed on the skin and rinsed off substantially immediately subsequent to application. The term “skin” as used herein includes the skin on the face (except eye lids and lips), neck, chest, abdomen, back, arms, under arms, hands, and legs. Preferably the term “skin” includes the skin on the face (except eye lids and lips) and under arms. More preferably it means skin on the face other than lips and eyelids.

The composition can be formulated in any known format, more preferred formats being creams or lotions.

Packaging for the composition of this invention can be a jar or tube as well as any other formats typically seen for cosmetic, cream, washing and lotion type products. The compositions may be applied topically and preferably 1-4 milligrams of composition is applied per square centimeter of skin.

Preferably, the composition is capable of delivering a pore CWA reduction of at least 35%, more preferably 40 to 80%, measured using the procedure in Examples.

The composition of the invention preferably delivers a cosmetic benefit to the skin of an individual to which it is topically applied. Examples of cosmetic benefits include reducing the appearance of fine lines, wrinkles, pores and/or blemish spots; evening skin tone, long lasting optical effect or a combination thereof on the desired skin surface.

The present invention also provides a method of improving the appearance of skin by blurring the imperfections of the skin comprising the steps of applying a composition as per the present invention on the desired skin surface.

The present invention further provides a use of a of the present invention for blurring benefit.

The present invention further provides the use of the composition as mentioned above wherein the blurring benefit is provided for reducing the appearance of fine lines, wrinkles, pores and or blemish spots on the skin.

The following examples are provided to facilitate an understanding of the invention. The examples are not intended to limit the scope of the claims.

According to present invention, there is provided a method for improving the appearance of skin by blurring the imperfections on the skin. The method comprises the step of applying a composition according to the first aspect on the desired skin surface.

According to present invention, there is provided a use of a composition according to the first aspect for blurring benefits. The blurring benefits is reflected by reduction of the appearance of fine lines, wrinkles. Pores and or blemish spots on the skin.

According to present invention, there is provided a use of a composition comprising titanium di-oxide and 8 to 60% by weight of a cross-polymer comprising adipic acid and neopentyl glycol is to provide blurring benefit the skin.

The present invention will now be demonstrated by way of non-limiting examples below. The examples are for illustration only and do not limit the scope of the invention in any manner.

### Examples

### Materials:

TABLE 1

| | | | Size |
|---|---|---|---|
| Material | Tradename | Supplier | (micron) |
| Titanium dioxide | SA-TR-10 | Miyoshi Kasei | 0.4 |
| Cross-polymer of | Halosphere ™ | Dermatech | NA |
| adipic acid and | N | | |
| neopentyl glycol | | | |
| Titanium dioxide | Chione ™ | BASF | 10-36 |
| Coated with mica | Snowfall | | |
| | White S130D | | |

### Effect of the Combination of Titanium Dioxide Particle and Crosspolymer of Adipic Acid and Neopentyl Glycol on the Blurring Efficacy.

A series of skin care formulations were prepared according to the table below:

TABLE 2

| | Examples (% w/w) | | | | | |
|---|---|---|---|---|---|---|
| Ingredients | A | B | C | D | E | 1 |
| Glycerine | 0.5 | 0.5 | 0.5 | 0.5 | 0.5 | 0.5 |
| Halosphere ™ | 50 | 50 | 0 | 50 | 10 | 50 |
| N* | | | | | | |
| SA-TR-10 | 0 | 0 | 0.4 | 0 | 0.4 | 0.4 |
| Titania dioxide | 0.4 | 0.8 | 0 | 0 | 0 | 0 |
| coated mica | | | | | | |
| Thickener | 1.25 | 1.25 | 1.25 | 1.25 | 1.25 | 1.25 |
| Cyclopenta- | 1.5 | 1.5 | 1.5 | 1.5 | 1.5 | 1.5 |
| siloxane | | | | | | |
| Sunscreens | 0.5 | 0.5 | 0.5 | 0.5 | 0.5 | 0.5 |
| Preservative | 0.025 | 0.025 | 0.025 | 0.025 | 0.025 | 0.025 |
| Water | to 100 | to 100 | to 100 | to 100 | to 100 | to 100 |

*Halosphere ™ N contains about 47% of cross polymer of adipic acid and neopentyl glycol

Example 1 is the formulation according to invention. Examples A to E are comparative examples.

### Method for Estimating Contrast Weighted Area (CWA):

The performance of the personal care compositions in Table 1 above was measured using the procedure as given below:

The image analysis algorithm was developed to extract the contrast between the pores and the background of BSP. The parameter of pore CWA, which calculates the contrast per image area for the pores, was used to measure and quantify the pore level. The simplest quantitative definition of contrast is the light intensity difference between a region of interest and its surroundings:

C=(L_{b}−L_{f})

Where C is the contrast, L_{f} is the light intensity (RGB value) of the pore, and L_{b} is the light intensity (RGB value) of the background. The higher pore CWA, the more visible pores. Thus, the pore CWA reduction is given by the following equation. The higher percentage of pore reduction, the better product efficacy.

$$\# Pore CWA reduction = \frac{Pore {CWA}_{baseline} - {CWA}_{product}}{Pore {CWA}_{baseline}} \times 100 \%$$

The commercial deep pore version of BioSkin plates (BSP) procured from Beaulax Co. Ltd., Japan (Code 10AN) were used as the in vitro substrate mimicking the appearance of human skin. The code 10AN representing sufficiently big pore size distribution. The above prepared formulations were then applied on this BSP with dosage of 2 mg/cm². It was then gently spread with finger cot, and then dried for 30 minutes at ambient temperature (˜25° C.), prior to taking images.

One image was taken before applying the formulations, denoted as “bef”. Another image was taken after applying the respective formulations, denoted as “aft”. The “% change from baseline”, calculated as “(bef−aft)/bef*100%”, which is also % pore Contrast weight average (CWA) reduction as for the attributes of pore CWA. Which is nothing but denotes the blurring efficacy of a formulation. The higher the pore CWA reduction, the better the formulation is in delivering the blurring benefit.

The results are summarized below in Table 3.

TABLE 3

| | Pore CWA reduction (%) | |
|---|---|---|
| Examples | Average | Standard deviation (±) |
| A | 18.8 | 6.2 |
| B | 12.9 | 7.3 |
| C | 8.1 | 4.0 |
| D | 22.7 | 4.4 |
| E | 31.7 | 6.2 |
| 1 | 44.1 | 1.6 |

From the above table it is evident that the example within the scope of the present invention (Example 1) provides significantly higher percentages of pore CWA reduction compared to comparative examples A to E. It is further evident from the above data (Example A and B) that titanium dioxide which is coated with some other substance (e.g. mica) does not work in combination with cross-polymer of adipic acid and neopentyl glycol.

Therefore, from the above description it is evident that the present invention provides a personal care composition that renders blurring effect on skin and thereby improves the skin appearance of individuals.

1. A personal care composition comprising:
a) titanium di-oxide having a particle size in the range of 200 to 600 nm; and
b) 8 to 60% by weight of a cross-polymer comprising adipic acid and neopentyl glycol;
wherein particle size means the apparent volume median diameter (D50) in non-aggregated state.

2. The personal care composition according to claim 1, wherein the amount of titanium di-oxide is in the range of 0.01 to 5% by weight of the composition.

3. The personal care composition according to claim 2, wherein the amount of titanium di-oxide in the composition is in the range of 0.01 to 2% by weight of the composition.

4. The personal care composition according to claim 1, wherein the weight ratio of the cross-polymer to the titanium di-oxide is in the range of 3000:1 to 1:1.

5. The personal care composition according to claim 4, wherein the particle size of titanium di-oxide is in the range of 200 to 500 nm.

6. The personal care composition according to claim 1, wherein the titanium di-oxide is coated with a hydrophobic coating.

7. The personal care composition according to claim 1, wherein the amount of cross-polymer is in the range of 12 to 60% by weight of the composition.

8. The personal care composition according to claim 1, additionally comprising one or more organic sunscreens.

9. A method of improving the appearance of skin by blurring the imperfections of the skin comprising the steps of applying the personal care composition according to claim 1, on the desired skin surface.

10. (canceled)

11. (canceled)

12. (canceled)

13. The personal care composition according to claim 1, wherein the weight ratio of the cross-polymer to the titanium di-oxide is in the range of 200:1 to 20:1.

14. The personal care composition according to claim 1, wherein the weight ratio of the cross-polymer to the titanium di-oxide is in the range of 120:1 to 30:1.

15. The personal care composition according to claim 4, wherein the particle size of titanium di-oxide is in the range of 200 to 400 nm.

16. The personal care composition according to claim 1, wherein the amount of cross-polymer is in the range of 18 to 30% by weight of the composition.

17. The personal care composition according to claim 8, wherein the organic sunscreen comprises 2-ethylhexyl-p-methoxycinnamate, butylmethoxydibenzoylmethane, or a mixture thereof.

18. The personal care composition according to claim 8, wherein the amount of organic sunscreen is in the range of 0.1 to 10%.

19. The personal care composition according to claim 1, wherein the composition further comprises a skin lightening agent.
